(19) United States
(12) Patent Application Publication
(10) Pub. No.: US 2008/0299056 A1
(43) Pub. Date: Dec. 4, 2008

(54) Passivated nano-titanium dioxide particles and methods of making the same

(76) Inventors: Scott Rickbeil Frerichs, Smyrna, DE (US); William Harvey Morrison, JR., Wilmington, DE (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(21) Appl. No.: 11/824,620

(22) Filed: Jul. 2, 2007

Related U.S. Application Data
(63) Continuation of application No. 10/737,357, filed on Dec. 16, 2003, now abandoned.

(51) Int. Cl. A61K 8/02; B01J 13/22; A61Q 17/04

(52) U.S. Cl. 424/59; 427/213.32

(57) ABSTRACT

The invention is directed to a method for reducing the chemical activity and photo activity of titanium dioxide nanoparticles comprising adding a densifying agent, such as citric acid, to an aqueous slurry of the titanium dioxide nanoparticles; treating the aqueous slurry with a source of silica, such as a solution of sodium silicate, to form silica treated titanium dioxide nanoparticles; treating the silica treated titanium dioxide nanoparticles with a source of alumina, such as a solution of sodium aluminate, to form silica and alumina treated titanium dioxide nanoparticles. The titanium dioxide nanoparticles described herein are useful in cosmetic, coating and polymer compositions.

### CROSS-REFERENCES TO RELATED APPLICATION

This application claims the benefit of U.S. patent application Ser. No. 10/737,357, filed Dec. 16, 2003, which is incorporated by reference in its entirety.

### FIELD OF THE INVENTION

The present invention relates to nanoparticle titanium dioxide compositions. More specifically, the invention relates to nanoparticle titanium dioxide particles which are silica and alumina treated in the presence of citric acid.

### BACKGROUND OF THE INVENTION

The scientific and technological advantages of nanostructured particles and materials have been attracting considerable attention. The small size of nanoparticles (generally used to indicate particles less than 100 nm in diameter), which can be responsible for different useful properties (electronic, optical, electrical, magnetic, chemical, and mechanical), makes them suitable for a wide variety of industrial applications.

Titanium dioxide (TiO₂) nanoparticles are substantially transparent to visible light but can absorb and scatter ultraviolet light. Titanium dioxide has low toxicity and is non-irritating to the skin. TiO₂ nanoparticles are especially advantageous when added to products in which transparency to visible light is important but exposure to the degrading and harmful effects of ultraviolet light is a problem. Such applications include, without limit, cosmetics, sunscreens, protective coatings, such as clear coatings for exterior wood and automobiles, and plastics.

Titanium dioxide itself is known to be photoactive. Free radicals form on the surface of the titanium dioxide particle under the action of ultraviolet rays. While the photoactivity of titanium dioxide is beneficial for use of titanium dioxide in photo catalyzed reactions, in other uses the free radicals can lead to degradation reactions and yellowing which can be disadvantageous. Such other uses include, without limit, cosmetics, sunscreens and plastics, wood and automotive coatings, etc. Thus, there is a desire for techniques that can photo-passivate the titanium dioxide; that is, render the titanium dioxide more photostable.

Untreated titanium dioxide nanoparticles are known to be chemically reactive. Untreated titanium dioxide will form highly colored complexes with certain antioxidants, such as ascorbic acid and ascorbic acid 6-palmitate. These colored complexes limit the use of titanium dioxide nanoparticles in applications where white creams and lotions are desired, such as cosmetics and sunscreens. Effective methods for passivation of the chemical reactivity of titanium dioxide nanoparticles are therefore desired. Thus, there is a desire for techniques that can make titanium dioxide nanoparticles nonreactive to such antioxidants.

Titanium dioxide nanoparticles are often prepared and/or used as a dispersion of the particles in a fluid medium, where the dispersion is, for example, an emulsion, slurry, cream, lotion or gel. However, dry titanium dioxide nanoparticles can form agglomerates and be difficult to disperse. Consequently, there is a need for titanium dioxide nanoparticles that are photopassived, have a reduced tendency to form agglomerates, and are easy to disperse in a fluid medium.

### BRIEF SUMMARY OF THE INVENTION

The present invention relates to a process for passivating titanium dioxide nanoparticles. The process of the invention comprises
(a) forming a slurry of titanium dioxide nanoparticles;
(b) contacting the slurry of titanium dioxide nanoparticles with a densifying agent;
(c) forming silica-coated titanium dioxide nanoparticles by contacting the slurry of step (b) with a silica source under conditions sufficient to deposit silica onto the titanium dioxide nanoparticles in an amount ranging from about 4 weight percent to about 18 weight percent based on the weight of the titanium dioxide nanoparticles in the mixture;
(d) forming an alumina coating on the silica-coated titanium dioxide nanoparticles by contacting the silica-coated titanium dioxide particles with an alumina source under conditions sufficient to deposit alumina in an amount ranging from about 5 weight percent to about 18 weight percent based on the weight of the titanium dioxide nanoparticles; and
(e) curing the titanium dioxide nanoparticles formed in step (d) to form silica- and alumina-coated titanium dioxide nanoparticles.

The invention also relates to a method for passivating titanium dioxide nanoparticles comprising contacting an aqueous slurry of the titanium dioxide nanoparticles with a densifying agent; treating the aqueous slurry with a solution comprising a silica source to form silica-treated titanium dioxide nanoparticles; treating the silica-treated titanium dioxide nanoparticles with a solution comprising an alumina source to form silica- and alumina-treated titanium dioxide nanoparticles.

The process of the instant invention has been found to produce titanium dioxide nanoparticles which are passivated as indicated by a high photo stability and/or high chemical stability. In addition the nanoparticles have a reduced tendency to form agglomerates.

### DETAILED DESCRIPTION OF THE INVENTION

The present invention provides titanium dioxide nanoparticles which are surface treated with amorphous silica and amorphous alumina in the presence of a densifying agent. More specifically, the particles are coated sequentially in a wet treatment process with amorphous silica and amorphous alumina in the presence of a densifying agent.

In a typical process of this invention, a slurry of titanium dioxide nanoparticles is heated and densifying agent is added to the slurry. The slurry is an aqueous mixture of the titanium dioxide particles, which are water insoluble. The slurry is then pH adjusted to form a basic composition and then treated with a source of silica, typically sodium silicate. The pH is decreased to a more neutral level by addition of acid, after which the slurry is treated with a source of alumina, typically sodium aluminate. After treatment with the source of silica and alumina the slurry is held at a certain pH and elevated temperature for a period of time sufficient to cure the particles. An objective of the curing step is to deposit silica and alumina onto the particles, more specifically, by coating the particles with a layer of silica and a layer of alumina.

The silica treatment occurs in the presence of a densifying agent. The densifying agent is important for densifying the coatings of silica and the alumina. Suitable densifying agents include citric acid and source of sulfate ion. Citric acid is the preferred densifying agent because of its dispersion enhancing properties. A useful amount of densifying agent is an amount sufficient to adequately densify the silica and alumina coatings. An excess of densification agent will maximize densification of the silica and alumina coatings but may lead to waste of the densifying agent. Suitable amounts of the densifying agent can be in the range of about 0.5% to about 3.0%, more typically from about 0.8% to about 2.4% based on weight of untreated TiO₂.

The concentration of TiO₂ in the slurry ranges from about 50 g/l to about 500 g/l more typically from about 125 to 250 grams per liter, although lower levels are also possible. Good coating consistency has been found with a relatively low concentration slurry. The temperature of the slurry usually ranges from about 45 to about 10° C. optimally from about 85 to about 100° C., although lower or higher temperatures might also be effective.

Pre-silica treatment, the slurry is maintained in the alkaline range, typically the pH is above 8.5, more typically 9.0 or higher although this may depend on the equipment used (lower pH may be possible for continuous wet treatment). The optimal silica deposition weight is typically between about 2 and about 20, more typically from about 5 to about 18% as SiO₂ based on weight of untreated TiO₂. However, improvements are likely to be seen at any silica level.

Any strong mineral acid, including HCl, HNO₃ and H₂SO₄ may be used to neutralize the slurry prior to alumina treatment. The optimal acid addition time for batch process ranges from 0.5 to about 4 minutes per 1% SiO₂ added. Longer times can lead to better product at the expense of rate.

The silica treated slurry is then held for a period of time sufficient to deposit a coating of silica on the titanium dioxide particles. The holding time is typically 5 minutes per 1% silica. Shorter times can be used but the coating may not be as effective. This holding step is typically carried out while maintaining a neutral to alkaline pH and elevated temperature. Thus, the pH usually is maintained at 7.0+1.0 and higher, typically up to and including about 10. The temperature is usually maintained above about 80° C., typically above about 9° C., more typically at about 95 to about 100° C.

In the alumina treatment the initial temperature of the slurry is optimally greater than about 80° C., typically above about 90° C., more typically in the range of about 95° C. to about 10° C., although lower temperatures might also be effective (or even more effective but at the expense of energy and time necessary to chill the slurry). Aluminate amount is optimally in the range of between about 5 and about 15% as Al₂O₃ based on weight of untreated TiO₂.

Any strong mineral acid can be employed during the alumina treatment including HCl, HNO₃, and H₂SO₄. The optimal acid addition time for batch process ranges from 0.5 to about 2.0 minutes per 1% Al₂O₃ added. Longer times can lead to better product at the expense of rate.

After adding the alumina, the pH of the slurry is typically held at a neutral level. Optimally at 7+0.5. Higher values might lead to undesired alumina phase; lower values to incomplete deposition.

The alumina treated slurry is then held for a period of time sufficient to form a coating of alumina on the titanium dioxide particles to which a silica coating has been deposited. The holding time is typically 3 minutes per 1% alumina. Shorter times can be used but the coating may not be as effective. This holding step, is typically carried out while maintaining a neutral pH and elevated temperature. Thus the pH usually is maintained at 7.0+0.5. The temperature is usually maintained at about 50° C., typically above about 45° C., more typically at about 55 to about 60° C.

Particulate compositions of the present invention generally include from about 2 to about 20, generally from about 5 to about 18% amorphous silica based on the weight of the untreated TiO₂ and from about 3 to about 20%, more typically from about 5 to about 15% amorphous alumina based on the weight of the untreated TiO₂.

The silica and alumina treated titanium dioxide nanoparticles, usually, are then filtered, washed and dried. The final particles are in a size range less than pigmentary; typically the average particle size is less than about 100 nanometers.

Any titanium dioxide nanoparticles can be suitable as a starting material for the process of this invention. As an example, titanium dioxide nanoparticles suitable as the starting material are described in U.S. Pat. Nos. 5,451,390; 5,672,330; and 5,762,914. Titanium dioxide P25 is an example of a suitable commercial product available from Degussa. Other commercial suppliers of titanium dioxide nanoparticles include Kemira and Tayca.

The titanium dioxide nanoparticle starting materials typically have an average particle size diameter of less than 100 nanometers (nm) as determined by dynamic light scattering which measures the particle size distribution of particles in liquid suspension. The particles are typically agglomerates which may range from about 3 nm to about 6000 nm. Any process known in the art can be used to prepare such particles. The process may involve vapor phase oxidation of titanium halides or solution precipitation from soluble titanium complexes, provided that titanium dioxide nanoparticles are produced.

A preferred process to prepare titanium dioxide nanoparticle starting material is by injecting oxygen and titanium halide, preferably titanium tetrachloride, into a high-temperature reaction zone, typically ranging from 400 to 2000 degrees centrigrade. Under the high temperature conditions present in the reaction zone, nanoparticles of titanium dioxide are formed having high surface area and a narrow size distribution. The energy source in the reactor may be any heating source such as a plasma torch. Optionally, the reactor may also include a flow homogenizer that ensures that feeds from the reactant inlets enter the reactor chamber downstream of the recirculation zone induced by the high temperature gas discharge. A flow homogenizer is described in U.S. Provisional Patent Application No. 60/434,158 filed on Dec. 17, 2002 which is incorporated herein by reference in its entirety.

The titanium dioxide starting material can be substantially pure titanium dioxide or may contain other inorganic material such as metal oxides. Examples include one or more of silica, alumina, zirconia and magnesia which can be incorporated into the particle using techniques known by those skilled in the art, for example these metal oxides can be incorporated when the titanium compounds are co-oxidized or co-precipitated with other metal oxide compounds. If such co-metals are present, they are preferably present in an amount of about 0.1 to about 5% based on the total metal oxide weight. The titanium dioxide starting material may also have one or more such metal oxide coatings applied using techniques known by those skilled in the art prior to treatment in accordance with this invention. In one embodiment of the invention, a slurry of substantially pure titanium dioxide is “pretreated” with alumina prior to contacting the slurry with citric acid. The pretreatment is typically to an amount of about 1 to about 4% based on the total metal oxide weight.

Typically, for alumina pretreated titanium dioxide, the final alumina level of products made by the invention is about 2.5% higher if the TiO₂ is pretreated with alumina.

Benefits have been found when the titanium dioxide nanoparticle starting material contains alumina, in a coating or by incorporation into the particle. For example, it has been found that the silica treatment step is more effective when applied to titanium dioxide particles that contain alumina. In addition, it has been found that the chemical stability (determined by the Vitamin C Yellowing Test which is described below) is higher and fewer oversized particles are produced by the process, specifically about 10% fewer oversized particles, as compared to a titanium dioxide starting material that does not contain alumina. By the term “oversized particles” it is meant agglomerates which are greater in diameter than about 200 nm, as determined by the MICROTRAC ultrafine particle analyzer.

The titanium dioxide starting material can also have an organic coating which may be applied using techniques known by those skilled in the art. A wide variety of organic coatings are known. Organic coatings employed for pigment-sized titanium dioxide may be utilized to coat nanoparticles. Examples of organic coatings that are well known to those skilled in the art include fatty acids, such as stearic acid; fatty acid esters; fatty alcohols, such as stearyl alcohol; polyols such as trimethylpropane diol or trimethyl pentane diol; acrylic monomers, oligomers and polymers; and silicones, such as polydimethylsiloxane and reactive silicones such as methylhydroxysiloxane. The final silica and alumina treated titanium dioxide particles of this invention may also be treated to have an organic coating.

Titanium dioxide nanoparticles made according to the present invention may be used with advantage in various applications including sunscreens and cosmetic formulations; coatings formulations including automotive coatings, wood coatings, and surface coatings; chemical mechanical planarization products; catalyst products; photovoltaic cells; plastic parts, films, and resin systems including agricultural films, food packaging films, molded automotive plastic parts, and engineering polymer resins; rubber based products including silicone rubbers; textile fibers, woven and nonwoven applications including polyamide, polyaramid, and polyimides fibers products and nonwoven sheets products; ceramics; glass products including architectural glass, automotive safety glass, and industrial glass; electronic components; and other uses in which photo and chemically passivated titanium dioxide nanoparticles will be useful.

One area of increasing demand for titanium dioxide nanoparticles is in cosmetic formulations, particularly in sunscreens as a sunscreen agent. Titanium dioxide nanoparticles provide protection from the harmful ultraviolet rays of the sun (UV A and UV B radiation). Both UV A and UV B radiation have been implicated in numerous skin problems, ranging from causing freckles, sunburn (erythema), and wrinkles, and premature aging. In addition, UV A radiation has been linked with skin cancer.

A dispersant is usually required to effectively disperse titanium dioxide nanoparticles in a fluid medium. Careful selection of dispersants is important. Typical dispersants for use with titanium dioxide nanoparticles include aliphatic alcohols, saturated fatty acids and fatty acid amines.

The titanium dioxide nanoparticles of this invention can be incorporated into a sunscreen formulation. Typically the amount of titanium dioxide nanoparticles can be unto about 25 wt. %, typically from about 0.1 wt. % to up to 15 wt. %, even more preferably unto 6 wt. %, based on the weight of the formulation, the amount depending upon the desired sun protection factor (SPF) of the formulation. The sunscreen formulations are usually an emulsion and the oil phase of the emulsion typically contains the UV active ingredients such as the titanium dioxide particles of this invention. Sunscreen formulations typically contain in addition to water, emollients, humectants, thickeners, UV actives, chelating agents, emulsifiers, suspending agents (typically if using particulate UV actives), waterproofers, film forming agents and preservatives.

Specific examples of preservatives include parabens. Specific examples of emollients include octyl palmitate, cetearyl alcohol, and dimethicone. Specific examples of humectants include propylene glycol, glycerin, and butylene glycol. Specific examples of thickeners include xanthan gum, magnesium aluminum silicate, cellulose gum, and hydrogenated castor oil. Specific examples of chelating agents include disodium ethylene diaminetetraacetic acid (EDTA) and tetrasodium EDTA. Specific examples of UV actives include ethylhexyl methoxycinnamate, octocrylene, and titanium dioxide. Specific examples of emulsifiers include glyceryl stearate, polyethyleneglycol-100 stearate, and ceteareth-20. Specific examples of suspending agents include diethanolamine-oleth-3-phosphate and neopentyl glycol dioctanoate. Specific examples of waterproofers include C30-38 olefin/isopropyl maleate/MA copolymer. Specific examples of film forming agents include hydroxyethyl cellulose and sodium carbomer. Numerous means are available for preparing dispersions of titanium dioxide nanoparticles containing dispersants. Intense mixing, such as milling and grinding may be needed, for example, to break down agglomerates into smaller particles. To facilitate use by the customer, producers of titanium dioxide nanoparticles may prepare and provide dispersions of the particles in a fluid medium which are easier to incorporate into formulations.

Because of the reduced photo activity of the titanium dioxide particles of this invention, they can be beneficial in products which degrade upon exposure to UV light energy.

Thus in one embodiment, the invention is directed to a coating composition suitable for protection against ultraviolet light comprising an additive amount suitable for imparting protection against ultraviolet light of the silica and alumina coated titanium dioxide nanoparticles made in accordance with this invention dispersed in a protective coating formulation.

Water based wood coatings, especially colored transparent and clear coatings benefit from a UV stabilizer which protects the wood. Organic UV absorbers are typically hydroxybenzophenones and hydroxyphenyl benzotriazoles. A commercially available UV absorber is sold under the trade name Tinuvin™ by Ciba. These organic materials, however, have a short life and decompose on exterior exposure. Replacing the organic material with titanium dioxide nanoparticles would allow very long lasting UV protection. The titanium dioxide passivated in accordance with this invention prevents the titanium dioxide from oxidizing the polymer in the wood coating, and is sufficiently transparent so the desired wood color can be seen. Because most wood coatings are water based, the titanium dioxide needs to be dispersible in the water phase. Various organic surfactants known in the art can be used to disperse the titanium dioxide nanoparticles in water.

Many cars are now coated with a clear layer of polymer coating to protect the underlying color coat, and ultimately the metal body parts. This layer has organic UV protectors, and like wood coatings, a more permanent replacement for these materials is desired. The titanium dioxide nanoparticles made in accordance with this invention are sufficiently transparent, and passivated for this application. The clear coat layers are normally solvent based, but can also be water based. Such coatings are well known in the art. The titanium dioxide nanoparticles can be modified for either solvent or water based systems with appropriate surfactants or organic surface treatments.

Titanium dioxide nanoparticles can be used to increase the mechanical strength of polymer composites. Most of these applications also require a high degree of transparency and passivation so underlying color or patterns are visible and the plastic is not degraded by the photoactivity of the titanium dioxide nanoparticles. The titanium dioxide nanoparticles must be compatible with the plastic and easily compounded into it. This application typically employs organic surface modification of the titanium dioxide nanoparticles as described herein above. The foregoing polymer composites are well known in the art.

### Test Methods

### Vitamin C Yellowing Test For Chemical Stability:

A standard solution of 6.25% ascorbic acid palmitate (L-ascorbic acid 6-palmitate, 99%, CAS #137-66-6, available commercially from Alfa Aesar) in octyl palmitate (hexadecanoic acid 2-ethylhexyl ester, CAS #29806-73-3, available under the name “Ceraphyl” by VanDyk) is prepared. Using a spatula and glass plate or Hoover Muller Model M5, 1.9+0.05 ml of the solution is thoroughly mixed with 0.4+0.01 g sample of titanium dioxide to be tested. The mixture is drawn down onto a white lacquered 3″×5″ card using a 6 mil Bird film applicator to form the test film. The color (L*a*b*) of the test film is measured using a hand-held spectrocolorimeter, such as Byk-Gardner Model CB-6805 which is warmed-up prior to taking the color reading, calibrated and set up to use D65/10 degree (illuminant/observer). In the same manner as the test film, a blank film is prepared using neat octyl palmitate and ultrafine titanium dioxide. The color of the blank film is measured in the same way as the color of the test film. The delta b* value is determined by comparing the color of the test and blank films. The delta b* value is a measure of chemical activity.

### UPA Particle Size Distribution

The MICROTRAC ULTRAFINE PARTICLE ANALYZER (UPA) (a trademark of Leeds and Northrup, North Wales, Pa.) uses the principle of dynamic light scattering to measure the particle size distribution of particles in liquid suspension. Leeds and Northrup, North Wales, Pa. manufacture the instrument. The measured size range is 0.003 μm to 6 μm (3 nm to 6000 nm). Use 2.55 for the refractive index of TiO₂ when setting up the UPA analysis. The dry particle sample needs to be prepared into a liquid dispersion to carry out the measurement. An example procedure is as follow:

(1) Weigh out 0.08 g dry powder.

(2) Add 79.92 g 0.1% tetra sodium pyrophosphate (TSPP) solution in water to make a 0.1 wt. % suspension.

(3) Sonify the suspension for 10 minutes using an ultrasonic probe. The suspension should be cooled in a waterjacketed beaker during sonication.

(4) When sonication is complete, draw an aliquot for analysis. Note, hydrophobic particles must first be wetted with a few drops of ethanol before adding into solution of TSPP.

The results of these tests were reported below for each of the examples.

### EXAMPLES

### Example 1

In a half gallon plastic jug containing 100 g nanometric titanium dioxide made by RF plasma oxidation according to US 2002/0155059A1 800 mls total volume deionized polished water was added and the mixture was stirred. The nanometric titanium dioxide starting material had a mean particle size of 90 nm, 10 wt % of particles less than 50 nm in size, and 90% of particles less than 150 nm in size as measured by the Microtrac UPA dynamic light scattering instrument. The mixture was sonicated for 10 minutes at a power of 7 and screened through a 325 mesh sieve. The screened mixture was added to a 2000 ml stainless steel beaker equipped with an electric stirrer, temperature probe and pH probe. The mixture was rapidly stirred using a propeller blade.

The initial pH was 1. The mixture was heated to 60° C. and the pH was adjusted to 7.1 with 50% NaOH solution (8.2 g). Then 9.0 g sodium aluminate (27.8 wt % alumina) was added. The pH was 10.8. The mixture was stirred for 15 minutes.

The mixture was heated to 92° C. The pH was 10.0. Then 1.6 g 50% citric acid solution was added. The pH after citric acid addition was 8.8. The pH was adjusted to 10.7 with 50% NaOH solution. Then 21.5 g sodium silicate (27 wt % silica) was added with strong stirring. The pH was 10.7. Over about 15 minutes concentrated (38%) hydrochloric acid solution was added to reduce the pH to 7 (17.7 g HCl). The mixture was stirred for 45 minutes at 92-95° C. The heat was stopped and the pH was reduced to the range of 6-8 with concentrated (38% HCl) (13.5 g) while adding 18.0 g sodium aluminate drop-wise over 15 minutes. The mixture was stirred for 20 minutes while maintaining a pH of 7. At the end of 20 minutes the temperature was 60. The pH was adjusted to 6.0±0.3 with concentrated (38%) HCl. The mixture was stirred again for 5 minutes. The final mixture was filtered, washed with deionized polished water to <143 mhos/cm conductance (˜3 liters water, 106 micro mhos/cm). The mixture was vacuum dried for about 30 minutes to form a cake then ethanol was added to cover the cake for about 15 minutes. The cake was then vacuum dried again for about 30 minutes. The cake was dried in a 125° C. oven on a tray overnight. The dry particles were ground and sieved through a 35 mesh screen and dried again.

Measured SiO₂: 3.9%

Measured Al₂O₃: 5.7%

### Example 2

The following materials were added to a 1000 ml plastic beaker: 50.00 g Degussa P25 titanium and 400 ml deionized polished water. The mixture was stirred then sonicated for 3 minutes at a power of 7. The mixture was then poured into a 600 ml stainless steel beaker equipped with an electric stirrer, temperature probe and pH probe. The mixture was agitated using a propeller blade. The initial pH of the mixture was 3.3. The mixture was heated to about 95° C. and 0.8 g citric acid 50% solution was added. The pH was 2.7. The pH was adjusted with 10% NaOH to a range of 9-9.5 by adding 3.8 g 50% NaOH solution. The neutral pH was maintained by adding 8.1 g concentrated (38%) HCl while adding 10.75 g sodium silicate drop wise over 14 minutes. The mixture was heated at 95° C. for one hour at pH 9.5 with stirring at about 2600 rpm. The pH was lowered to 7 by adding 8.1 g concentrated (38%) HCl while 9 g sodium aluminate was added drop wise over 10 minutes. The heat was turned off and the mixture was stirred for 20 minutes at pH of 7. The temperature after 20 minutes was 75.5° C. The pH was adjusted to 6.0±0.3 with HCl and stirred for 5 minutes.

The mixture was filtered, washed and dried and the dry particles were formed as in Example 1.

Measured SiO₂: 4.4%

Measured Al₂O₃: 3.2%

### Example 3

The treatment was performed as in Example 1 except no sodium aluminate was added prior to the addition of sodium silicate.

Measured SiO₂: 4.1%

Measured Al₂O₃: 4.4%

### Example 4

The aqueous mixture of titanium dioxide was prepared, stirred then solicated and pH adjusted as in Example 1. The initial pH was 1.5. The mixture was heated to 60° C. and the pH was adjusted to 7.3 with 50% NaOH solution (8.2 g). Then 9.0 g sodium aluminate (27.8 wt % alumina) was added. The pH was 11.4. The mixture was stirred for 15 minutes.

The mixture was heated to 92° C. The pH was 10.9. Then 4.8 g 50% citric acid solution was added. The pH after citric acid addition was 9.7. The pH was adjusted to 10.9 with 50% NaOH solution. Then 64.5 g sodium silicate (27 wt % silica) was added with strong stirring. The pH was 11.0. Over about 15 minutes concentrated (38%) hydrochloric acid solution was added to reduce the pH to 7 (23.5 g.HCl). The mixture was stirred for 45 minutes at 2-95° C. The heat was stopped and the pH was reduced to the range of 6-8 with concentrated (38% HCl)(37.4 g) while adding 54.0 g sodium aluminate drop-wise over 13 minutes. The mixture was stirred for 20 minutes while maintaining a pH of 7. At the end of 20 minutes the temperature was 44° C. The pH was adjusted to 6.0±0.3 with concentrated (38%) HCl The mixture was stirred again for 5 minutes. The final mixture was filtered, washed with deionized polished water to <143 mhos/cm conductance (˜3 liters water, 100 micro mhos/cm). The mixture was vacuum dried for about 30 minutes to form a cake then ethanol was added to cover the cake for about 15 minutes. The cake was then vacuum dried again for about 30 minutes. The cake was dried in a 125° C. oven on a tray overnight. The dry particles were ground and sieved through a 35 mesh screen and dried again.

Measured SiO₂: 10.1%

Measured Al₂O₃:14.5%

### Example 5

The aqueous mixture of titanium dioxide was prepared, stirred, sonicated and pH adjusted as in Example 1. It was then heated to 60° C. and stirred for 15 minutes, then filtered, washed, and dried as in Example 1.

Measured SiO₂: 0.0%

Measured Al₂O₃: 0.0%

### Example 6

The following materials were added to a 1000 ml plastic beaker: 50.00 g Degussa P25 titanium dioxide and 400 ml deionized polished water. The mixture was stirred then sonicated for 3 minutes at a power of 7. The mixture was then agitated with an electric stirrer motor and heated to 92° C. The initial pH was 3.2. The pH was adjusted to 9.2 using 1.4 g 10% NaOH. The pH of the mixture was maintained in a range of 9-10 using HCl (18%, 10.3 g, 50% dilute) while 18.5 g sodium silicate solution (27 wt. % SiO₂) was added drop wise over 8 minutes. The mixture was heated for one hour.

The mixture was filtered, washed and dried as described in Example 1 and the particles were ground and sieved through a mesh screen and dried again.

Measured SiO₂: 8.33%

### Example 7

The aqueous mixture of titanium dioxide was prepared, stirred then sonicated as described in Example 2. The initial pH was in the range of 3.3-3.6. The mixture was heated to about 91° C. The pH was adjusted to 9.4 using 1.24 g 10% NaOH. The pH of the mixture was maintained in a range of 9-9.5 using HCl (18%, 20.63 g, 50% dilute) while adding 37.04 g sodium silicate solution (27 wt. % SiO₂) drop wise over about 40 minutes. The mixture was heated to 91-97° C. for one hour at pH of 9.3.with mixing at about 2700 rpm.

The mixture was filtered, washed and dried as described in Example 1 and the particles were ground and sieved through a 100 mesh screen and dried again.

Measured SiO₂: 13.0%

### Example 8

The aqueous mixture of titanium dioxide was prepared, stirred then sonicated as described in Example 2. The initial pH was in the range of 3.4-3.8. The mixture was heated to 92° C. The pH was adjusted to 9.2 using 1.1 g 10% NaOH. The pH of the mixture was maintained in a range of 9-9.5 using HCl (38%, 39.20 g, 50% dilute) while adding 55.56 g sodium silicate solution (27 wt. % SiO₂) drop wise over about 27 minutes. The mixture was heated to 94° C. for one hour at pH of 9.4.with mixing at about 3500 rpm.

The mixture was filtered, washed and dried as described in Example 1 and the particles were ground and sieved through a 100 mesh screen and dried again.

Measured SiO₂: 20.0%

### Example 9

The aqueous mixture of titanium dioxide was prepared, stirred then sonicated as described in Example 2. The initial pH was in the range of 3.0-3.1. The mixture was heated to 92° C. The pH was adjusted to 9.1-9.5 using about 1.6 g 10% NaOH and maintained at that pH. The mixture was heated to 90-98° C. for one hour at pH of 9.5. The mixture was filtered, washed and dried as described in Example 1 but it was noted that filtering and washing was slower than Examples made with sodium silicate. The dried material had a tan color. The particles were ground and sieved through a 100 mesh screen and dried again.

Measured SiO₂=0%

Measured Al₂O₃=0%

### Example 10

The aqueous mixture of titanium dioxide was prepared, stirred then sonicated and pH adjusted as in Example 1. The mixture was heated to 60° C.

Then 27.0 g sodium-aluminate (27.8 wt % alumina) was added while keeping the pH in the range of 6-8 using 19.5 g of concentrated (38%) HCl. The mixture was then stirred for 20 minutes maintaining the pH and temperature.

The material was then filtered, washed, dried, and crushed as in Example 1.

Measured Al₂O₃=4.7%

TABLE 1

| Example | % SiO₂ | % Al₂O₃ | Delta b*¹ | PSD² |
|---|---|---|---|---|
| 1 | 3.9 | 5.7 | 1.7 | 54 |
| 2 | 4.4 | 3.2 | 3.5 | 50 |
| 3 | 4.1 | 4.4 | 5.4 | 65 |
| 4 | 10.1 | 14.5 | 1.0 | 61 |
| 5 | 0 | 0 | 27 | 13 |
| 6 | 8.3 | 0 | 17.6 | 45 |
| 7 | 13.0 | 0 | 12.6 | 61 |
| 8 | 20.0 | 0 | 4.3 | 85 |
| 9 | 0 | 0 | 25 | 32 |
| 10 | 0 | 4.7 | 23 | — |

¹As determined by the Vitamin C Yellowing Test
²As determined by the MICROTRAC UPA

The delta b* (an indication of chemical activity) values of Examples 6, 7 and 8 show that increasing the % silica lowers the delta b* values which indicates that higher levels of silica will lead to a more chemically stable product. However, as the silica content increases the particles have a greater tendency to form agglomerates, as indicated by the PSD values. Example 2 shows that titanium dioxide particles having silica and alumina coatings have a low delta b* value indicating good chemical stability especially in comparison to untreated material (Example 5) and, in addition, the agglomeration is substantially reduced, as indicated by the PSD values. Thus, silica and alumina coated titanium dioxide nanoparticles having low surface treatment levels have chemical stability properties which are as good as, if not better than, titanium dioxide particles that contain high silica levels. Examples 1, 2, and 3 show that this treatment can also be applied to a titanium dioxide nanoparticles formed by different processes with good effectiveness and produce chemically stable particles, especially compared to the untreated material (Example 5), that have reduced agglomeration compared to silica only treated particles (Example 8).

The description of illustrative and preferred embodiments of the present invention is not intended to limit the scope of the invention. Various modifications, alternative constructions and equivalents may be employed without departing from the true spirit and scope of the appended claims.

What is claimed is:

1. A process for passivating titanium dioxide nanoparticles comprising
(a) forming a slurry of titanium dioxide nanoparticles;
(b) contacting the slurry of titanium dioxide nanoparticles with a densifying agent;
(c) forming silica coated titanium dioxide nanoparticles by contacting the slurry of step (b) with a source of silica under conditions sufficient to deposit silica onto the titanium dioxide nanoparticles in an amount ranging from about 5 weight percent to about 18 weight percent based on the weight of the titanium dioxide nanoparticles in the mixture;
(d) forming an alumina coating on the silica coated titanium dioxide nanoparticles by contacting the slurry of step (c) with a source of alumina under conditions sufficient to deposit alumina in an amount ranging from about 5 weight percent to about 15 weight percent based on the weight of the titanium dioxide nanoparticles; and
(e) curing the titanium dioxide nanoparticles formed in step (d) to form silica and alumina coated titanium dioxide nanoparticles.

2. The process of claim 1 further comprising contacting the slurry of titanium dioxide nanoparticles with sodium aluminate prior to contacting the slurry with densifying agent.

3. The process of claim 1 in which the conditions of forming silica coated titanium dioxide particles comprise contacting the titanium dioxide particles with sodium silica at a pH of at least about 10 and elevated temperature.

4. The process of claim 1 in which the conditions of forming an alumina coating comprise contacting the titanium dioxide with sodium aluminate at a pH ranging from about 5 to 9 and an elevated temperature.

5. The process of claim 1 in which the densifying agent is added to the slurry to a concentration based on the weight of the titanium dioxide nanoparticles of from about 0.1 to about 3%.

6. The process of claim 1 further comprising contacting the silica and alumina coated titanium dioxide particles with an organic composition.

7. The process of claim 6 in which the organic composition comprises at least one of octyltriethoxysilane, aminopropyltriethoxysilane, polyhydroxystearic acid, and polyhydroxy siloxide.

8. The process of claim 1 in which the densifying agent is citric acid.

9. A composition for screening ultra violet radiation comprising silica and alumina coated titanium dioxide nanoparticles made by the process of claims 1, 6, 8 or 9 dispersed in an organic or aqueous medium.

10. A method for reducing the chemical activity and photo activity of titanium dioxide nanoparticles comprising contacting an aqueous slurry of the titanium dioxide nanoparticles with a densifying agent; treating the aqueous slurry with a source of silica to form silica treated titanium dioxide nanoparticles; treating the silica treated titanium dioxide nanoparticles with a source of alumina to form silica and alumina treated titanium dioxide nanoparticles.

11. The method of claim 10 in which the densifying agent is citric acid.
